(19) United States
(12) Patent Application Publication
(10) Pub. No.: US 2010/0076317 A1
(43) Pub. Date: Mar. 25, 2010

(54) CATHETER WITH ULTRASOUND TRANSDUCER AND VARIABLE FOCUS LENS USED IN ANEURYSM ASSESSMENT

(75) Inventors: Drazenko Babic, Best (NL); Jan Frederik Suijver, Dommelen (NL); Bernardus Hendrikus Wilhelmus Hendriks, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/516,617

(22) PCT Filed: Nov. 16, 2007
(86) PCT No.: PCT/IB07/54683

§ 371 Date: May 28, 2009

Related U.S. Application Data
(60) Provisional application No. 60/867,833, filed on Nov. 30, 2006.

(51) Int. Cl. A61B 8/14

(52) U.S. Cl. 600/466

(57) ABSTRACT

A catheter having ultrasound capability and a distal end extending along a longitudinal axis, the end being situated adjacent to a tissue of interest inside of a patient; at least one ultrasound transducer located in the end to direct ultrasound waves along the axis in a generally forward direction relative to the end; a variable-focus lens system located in the end, downstream of the transducer in the generally forward direction, the lens system being capable of variably focusing the ultrasound waves emitted by the transducer at various positions downstream of the lens and catheter end; optionally, a mirror located in the end, downstream of the transducer and the lens system in the generally forward direction, whereby the emitted ultrasound waves from the lens system are either reflected from the mirror and focused at a position substantially perpendicular to the longitudinal axis; or the emitted ultrasound waves from the lens system substantially bypass the mirror in unreflected form and are focused at a position downstream of the transducer, the lens system and the mirror in the generally forward direction along the longitudinal axis; and imaging means for translating the focused ultrasound waves from the lens system reflected and/or unreflected by the mirror into three-dimensional images and communicating the images in viewable form to a human operator that is external to the patient. A method of measuring, imaging and viewing blood flow velocity and patterns in a blood vessel and aneurysm, as well as monitoring vaso-occlusive coil placement in an aneurysm during a surgical intervention using the catheter, is also disclosed.

The invention relates to a catheter having an ultrasound transducer and variable focus lens system located in the catheter end, and method of use in providing dynamic 3D imaging and blood flow measurements in a tissue of interest, for example a blood vessel and aneurysm, and monitoring during a surgical intervention of the proper placement of vaso-occlusive wire coils into the aneurysm cavity.

Aneurysms are caused by weakening of the arterial wall that finally results in a bulge formation in different appearance shapes. Although the anatomical and physiological origin of the bulge formation is not thoroughly understood, there is extensive evidence that alterations in blood flow pattern cause aneurysm formation.

When the wall of such an aneurysm weakens it can rupture, leading to hemorrhaging. When this occurs in the brain it will lead to a stroke, which can have fatal consequences. Typically, interventional physicians will operate immediately upon detection of an aneurysm. However, a large fraction of the (especially the elder) population have asymptomatic aneurysms, whereby the aneurysm has stabilized resulting in a low risk for the patient. In such cases, treatment of the aneurysm is not advised, as the treatment itself significantly increases the probability for stroke.

Aneurysm formation starts by intima layer damages in the arterial wall, caused by blood flow shear stress. Prolonged shear stress causes modification and break down of the intima cells. The shear stress depends upon the vessel geometry, blood viscosity, cardiac cycle phase, blood viscosity, blood speed etc.

There are specific places on the arterial wall that are more prone to shear stress than others due to the vessel geometry (curved vessel portions, bifurcations and trifurcations). After an aneurysm formation has taken place, blood flow pattern within the aneurysm pouch is of a crucial importance to predict growth pattern and rupture occurrence.

Assessment of the blood flow as a predictor of the aneurysm formation and growth as well as the dynamic assessment of the blood flow inside the aneurysm pouch is crucial in order to understand and predict aneurysm behavior. Well-understood and clinically proven and reproducible flow assessment would potentially improve vascular interventionalist's or vascular interventional physician's ability to define the most optimal treatment strategy.

The fundamental question here is, how does one determine whether or not intervention in a particular patient is required. This translates into the problem of determining the blood flow velocity inside the artery as well as in the aneurysm. From the comparison of these values, as well as their fluctuations over time, an assessment of the risk involved with a particular aneurysm can be made.

There are several techniques that have previously been disclosed in order to assess the intracranial blood flow and intraaneurysmal flow pattern, but none of which has met the clinical requirements so far:

MR blood flow quantification method that suffers from various technical shortcomings: low spatial resolution (inability to visualize small vessels and small aneurysms), inability to cope with turbulent blood flow (missing vessels and missing aneurysms) etc.

CT gated multi-slice scanning combined with various blood flow simulation techniques. The latter is based on computational fluid dynamic methods that are able to simulate blood flow and predict shear stress level at various stages of cardiac cycle. However, this technique is not base on real-time endovascular measurements of the aneurysm flow pattern, which often results in erroneous interpretation of the imaging results. Furthermore, the spatial resolution issues as well as discontinuous movements that are not synchronized with the heart rate are often limiting factors as well.

Trans-cranial Doppler imaging—provides real time blood flow assessment of the extracranial arteries and small supply territory of the middle cerebral artery. However, this technique is not considered as clinically relevant in the intracranial blood flow detection.

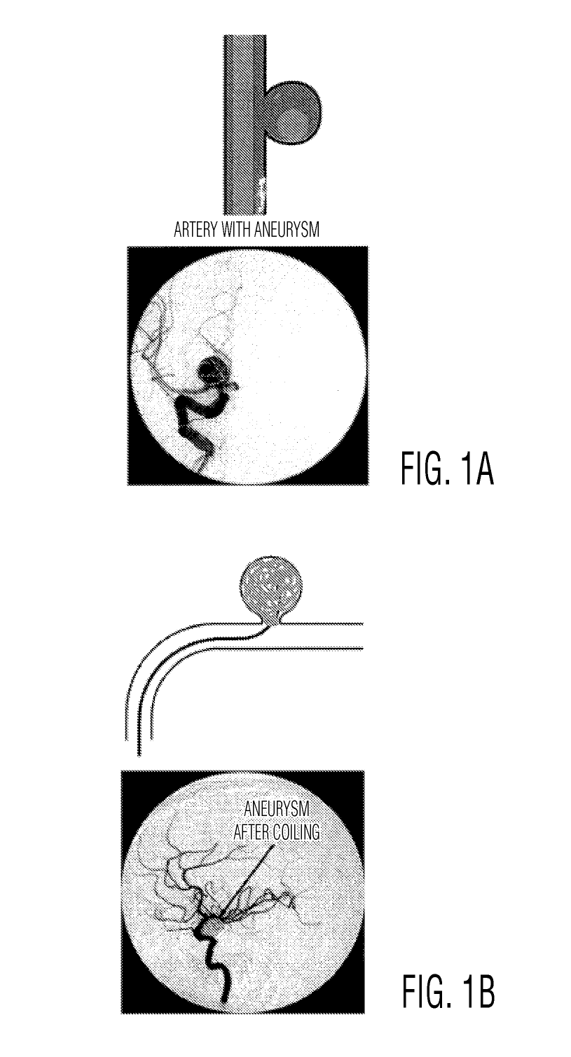

Additionally, in treating aneurysms, delivery or placement of embolization material, such as endovascular or vaso-occlusive wire coils within the intracranial cavity or pouch of the aneurysm has proven to be an effective and safe medical treatment. The technique is based on intraarterial access of the aneurysm pouch to be treated by the means of coils that are delivered inside the vascular pathology. The technique, particularly using platinum made wire coils, is considered as a gold standard in the treatment of the various types of intra and extracranial aneurysms worldwide, and is schematically depicted in FIG. 1.

The major concern in the treatment of the aneurysms is a dislocation of the coils during or after the treatment as well as aneurysm sack re-exposure to the blood flow that may eventually result in a recurrent hemorrhage. One of the most important issues to be solved during the treatment is to achieve a high compaction rate of the coils, which means as good packing rate as possible (by decreasing the space between the coil loops). A high compaction rate will finally decrease the so-called water hammer effect of the pulsatile blood flow and as such lower the risk of rebleeding.

Due to the poorly defined decision-making process, interventional physicians do not know when the appropriate number of coils has been introduced into the aneurysm, and they tend to place too many rather than too few. At a price point of roughly US$ 1000,—per coil, this represents a significant waste of resources. As an example: patients with over 100 coils in a single aneurysm are not an exception, even though as few at 10 may already have been enough.

Clearly, the problem here is to determine at which point enough coils have been introduced into the aneurysm.

The high package rate of the coils during the treatment is usually difficult to achieve due to multiple technical reasons:

Beam hardening artifacts in the X-ray angio machine caused by the high density material in the X-ray field of view

Magnification Artifacts

Non-existing evaluation method for 3D assessment of the placed coils

Difficulties in accurate determination of the aneurysm shape (especially in giant aneurysms) and its volume in relation to the volume of the coils placed inside the aneurysm

Non-existing evaluation of the aneurysm volume filled with thrombus

X-ray tube positioning artifacts (multiple superimposition of coils on 2D projection images during the intervention)

In order to solve these issues, several techniques have been developed in the mean time:

3D assessment of the aneurysm shape and 3D volumetric analysis of the aneurysm pouch 3D assessment of both the aneurysm and inserted coils

Although the mentioned techniques have improved the state of the art for endovascular treatment of the aneurysms, there are still several unsolved issues that are presented below:

inaccurate 3D assessment of the aneurysms filled with coils in terms of aneurysm and coils volume—the previously mentioned beam hardening artifacts, caused by high X-ray attenuation material, results in very poor 3D visualization of the coils (very global visualization of the outer boundary of the coil mash). That means that peri- and post-procedural assessment of the 3D coils placement is not clinically applicable.

The coils compaction rate is visually roughly estimated on the basis of the blood flow obstruction within the aneurysm, assessed by intraprocedural angiograms that are repeated after every coil placement. This assessment is considered to be very subjective and therefore unreliable.

The coil position with respect to the aneurysm neck is hardly achieved at all—multiple superimposition of coil loops in 2D X-ray projection images do not allow for 3D understanding of the outer location of the coils with respect to the aneurysm neck. Erroneous positioning of the coils can potentially cause dislocation of the coils from the aneurysm pouch to the parent vessel blood stream that often results in blood flow obstruction distally to the aneurysm with fatal consequences for the patient.

However, problems still persist with these systems and methodology for providing dynamic, accurate, real time three-dimensional (3D) imaging of the tissues of interest inside a patient, particularly with respect to internal vasculature, for example, blood vessels and aneurysms, and methods of monitoring proper placement of vaso-occlusive coils used in treating aneurysms, which the herein disclosed methodology and systems overcome.

According to this invention, herein disclosed is a catheter having an ultrasound transducer and variable focus lens system located in the catheter end, and method of use in providing dynamic 3D imaging and blood flow measurements in a tissue of interest, for example a blood vessel and aneurysm, and monitoring during a surgical intervention of the proper placement of vaso-occlusive wire coils into the aneurysm cavity.

Specifically, it is an object of the invention to provide a catheter having ultrasound capability and a distal end extending along a longitudinal axis, the end being situated adjacent to a tissue of interest inside of a patient, the catheter comprising:

at least one ultrasound transducer located in the end to direct ultrasound waves along the axis in a generally forward direction relative to the end;

a variable-focus lens system located in the end, downstream of the transducer in the generally forward direction, the lens system being capable of variably focusing the ultrasound waves emitted by the transducer at various positions downstream of the lens and catheter end;

optionally, a mirror located in the end, downstream of the transducer and the lens system in the generally forward direction, whereby the emitted ultrasound waves from the lens system are either reflected from the mirror and focused at a position substantially perpendicular to the longitudinal axis; or the emitted ultrasound waves from the lens system substantially bypass the mirror in unreflected form and are focused at a position downstream of the transducer, the lens system and the mirror in the generally forward direction along the longitudinal axis; and

imaging means for translating the focused ultrasound waves from the lens system reflected and/or unreflected by the mirror into three-dimensional images and communicating the images in viewable form to a human operator that is external to the patient.

Another object is to provide a catheter wherein the lens system comprises two immiscible liquids that form a boundary between the liquids, and means for applying a force directly onto at least a part of one of the liquids so as to selectively induce a displacement of part of the boundary and thereby vary the focal point of the lens system.

Another object is to provide a catheter wherein the mirror is positioned in a plane that is at a 45 degree angle to the longitudinal axis and of a smaller size relative to the size of the lens system.

Another object is to provide a catheter wherein the tissue of interest is a blood vessel having an aneurysm, the endoscope further comprising a Doppler ultrasound velocity measuring means for measuring the velocity of blood flow at one or more locations in the vessel and/or the aneurysm and communicating in viewable form the measured blood flow velocity and blood flow pattern information to a human operator that is external to the patient.

Another object is to provide a catheter further comprising embolization material delivery means for delivering embolization material into the cavity of the aneurysm, wherein the embolization material is capable of causing an occlusion to form within the aneurysm cavity and substantially prevent further blood flow into the aneurysm cavity.

Another object is to provide a catheter wherein the embolization material comprises at least one vaso-occlusive wire coil.

Another object is to provide a catheter wherein the coil is made of platinum.

Another object is to provide a method of viewing a tissue of interest inside of a patient comprising:

disposing inside the patient a catheter having ultrasound capability and a distal end extending along a longitudinal axis, the end being situated adjacent to a tissue of interest inside of a patient, the catheter comprising:

at least one ultrasound transducer located in the end for transmitting ultrasound waves along the axis in a generally forward direction relative to the end, and for receiving resultant echoes of the ultrasound waves reflected by the tissue of interest;

a variable-focus lens system located in the end, downstream of the transducer in the generally forward direction, the lens system being capable of variably focusing the ultrasound waves emitted by the transducer at various positions downstream of the lens and catheter end;

optionally, a mirror located in the end, downstream of the transducer and the lens system in the generally forward direction, whereby the emitted ultrasound waves from the lens system are either reflected from the mirror and focused at a position substantially perpendicular to the longitudinal axis; or the emitted ultrasound waves from the lens system substantially bypass the mirror in unreflected form and are focused at a position downstream of the transducer, the lens system and the mirror in the generally forward direction along the longitudinal axis; and

imaging means for translating the received echoes of the ultrasound waves reflected by the tissue of interest into three-dimensional images and communicating the images in viewable form to a human operator that is external to the patient;

transmitting ultrasound waves from the transducer;

variably focusing the emitted ultrasound waves with the lens system at various positions of the tissue of interest; and

receiving by the transducer of the resultant echoes of the ultrasound waves; and

translating the received echoes of the ultrasound waves into three-dimensional images and communicating the images in viewable form to a human operator that is external to the patient.

Another object is to provide a method wherein the lens system comprises two immiscible liquids that form a boundary between the liquids, and means for applying a force directly onto at least a part of one of the liquids so as to selectively induce a displacement of part of the boundary and thereby vary the focal point of the lens system.

Another object is to provide a method wherein the mirror is positioned in a plane that is at a 45 degree angle to the longitudinal axis and of a smaller size relative to the size of the lens system.

Another object is to provide a method further comprising measuring the blood flow velocity and blood flow pattern in a blood vessel and/or an aneurysm associated with the blood vessel, wherein the catheter further comprises a Doppler ultrasound velocity measuring means for measuring the velocity of blood flow at one or more locations in the vessel and/or the aneurysm and communicating in viewable form the measured blood flow velocity and blood flow pattern information to a human operator that is external to the patient.

Another object is to provide a method further comprising delivery of embolization material into the cavity of the aneurysm, wherein the catheter further comprises embolization material delivery means for delivering embolization material into the cavity of the aneurysm, the embolization material being capable of causing an occlusion to form within the aneurysm cavity and substantially prevent further blood flow into the aneurysm cavity.

Another object is to provide a method wherein the embolization material comprises at least one vaso-occlusive wire coil.

Another object is to provide a method wherein the coil is made of platinum.

Another object is to provide a method wherein the tissue of interest is a blood vessel, the catheter further comprising a Doppler ultrasound velocity measuring means for measuring the velocity of blood flow at one or more locations in the vessel and communicating in viewable form the measured blood flow velocity and blood flow pattern information to a human operator that is external to the patient.

Another object is to provide a method of monitoring during a surgical intervention the placement of one or more vaso-occlusive wire coils in a blood vessel aneurysm inside a patient, the method comprising:

disposing inside the patient a catheter having ultrasonic capability, coil delivery capability and a distal end extending along a longitudinal axis, the end being situated inside the blood vessel and adjacent to the aneurysm, the catheter comprising:

at least one ultrasound transducer located in the end for transmitting ultrasound waves along the axis in a generally forward direction relative to the end, and for receiving resultant echoes of the ultrasound waves reflected by the aneurysm and blood vessel;

a variable-focus lens system located in the end, downstream of the transducer in the generally forward direction, the lens system being capable of variably focusing the ultrasound waves emitted by the transducer at various positions downstream of the lens and catheter end;

optionally, a mirror located in the end, downstream of the transducer and the lens system in the generally forward direction, whereby the emitted ultrasound waves from the lens system are either reflected from the mirror and focused at a position substantially perpendicular to the longitudinal axis; or the emitted ultrasound waves from the lens system substantially bypass the mirror in unreflected form and are focused at a position downstream of the transducer, the lens system and the mirror in the generally forward direction along the longitudinal axis;

imaging means for translating the received echoes of the ultrasound waves reflected by the aneurysm and blood vessel into three-dimensional images and communicating the images in viewable form to a human operator that is external to the patient;

Doppler ultrasound velocity measuring means for measuring the blood flow velocity and blood flow pattern in a blood vessel and/or an aneurysm associated with the blood vessel, wherein the catheter further comprises a Doppler ultrasound velocity measuring means for measuring the velocity of blood flow at one or more locations in the vessel and/or the aneurysm and communicating in viewable form the measured blood flow velocity and blood flow pattern information to a human operator that is external to the patient; and

coil delivery means for delivering one or more coils into the cavity of the aneurysm, wherein the one or more coils are capable of causing an occlusion to form within the aneurysm cavity and substantially prevent further blood flow into the aneurysm cavity;

transmitting ultrasound waves from the at least one transducer;

variably focusing the emitted ultrasound waves with the lens system at various positions of the blood vessel and aneurysm cavity; and

receiving by the at least one transducer of the resultant echoes of the ultrasound waves and blood velocity measurements; and

translating the received echoes of the ultrasound waves and blood velocity measurements into three-dimensional images and communicating the images in viewable form to a human operator that is external to the patient to enable the operator to monitor the proper placement of the coils in the aneurysm cavity.

FIG. 1 is a schematic representation of (top left) a blood vessel or artery with an aneurysm, (top right) a catheter entering the aneurysm and delivering a series of platinum coils, (bottom left) an X-ray image of an aneurysm directly after coiling, (bottom right) an X-ray image of an aneurysm several days after coiling when the aneurysm has been neutralized.

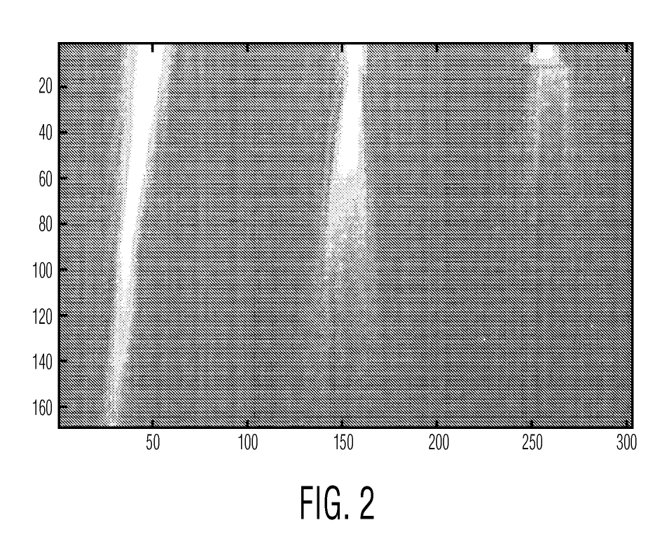

FIG. 2 shows examples of beam steering (left), no influence (middle), and focusing (right) of ultrasound with a variable focus lens. This data was recorded with a 5 MHz ultrasound transducer and a 0.25 cm radius circular variable focus lens (silicone oil/water). The values on the axis are in millimeters.

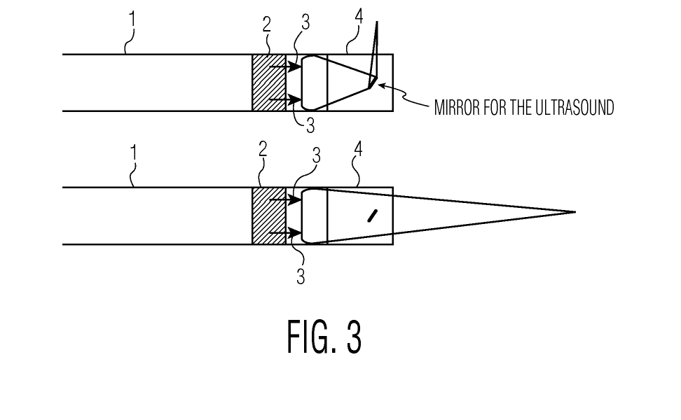

FIG. 3 is a schematic representation of an embodiment of the invention. Top figure: focusing perpendicular to the longitudinal axis of the catheter. Bottom figure: focusing into the catheter direction along its longitudinal axis.

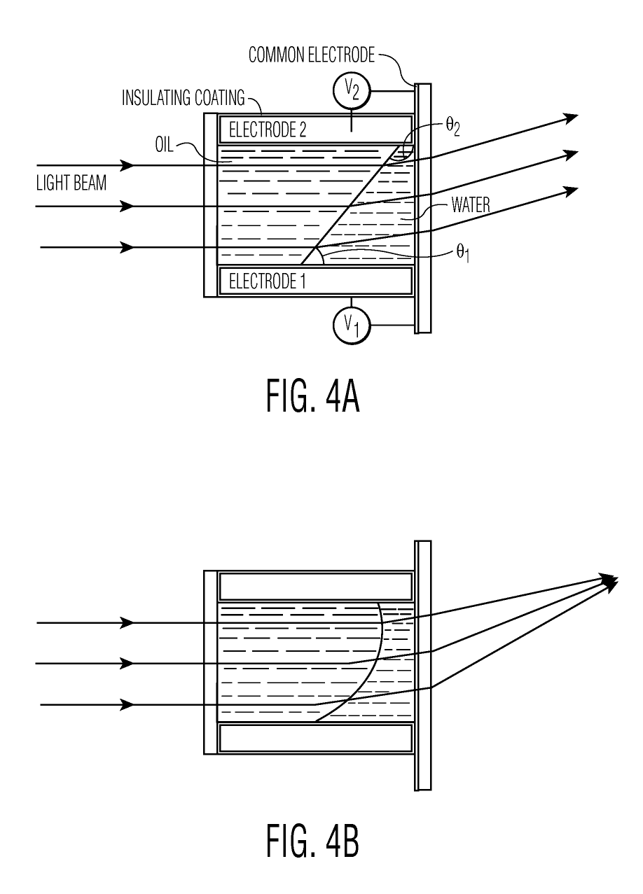

FIG. 4 shows an example of a variable focus lens where the meniscus can be curved and tilted, allowing ultrasound focusing and beam steering.

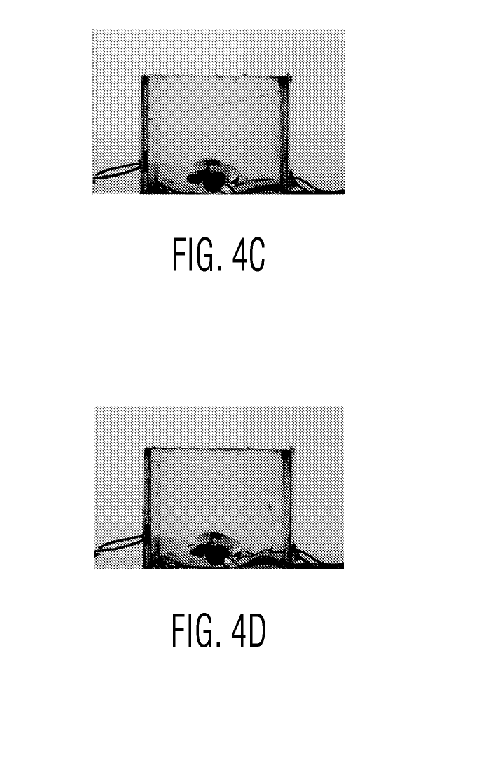

FIG. 5 shows a schematic drawing of the tip of the catheter containing a transducer and a variable focus lens capable of ultrasound steering and focusing.

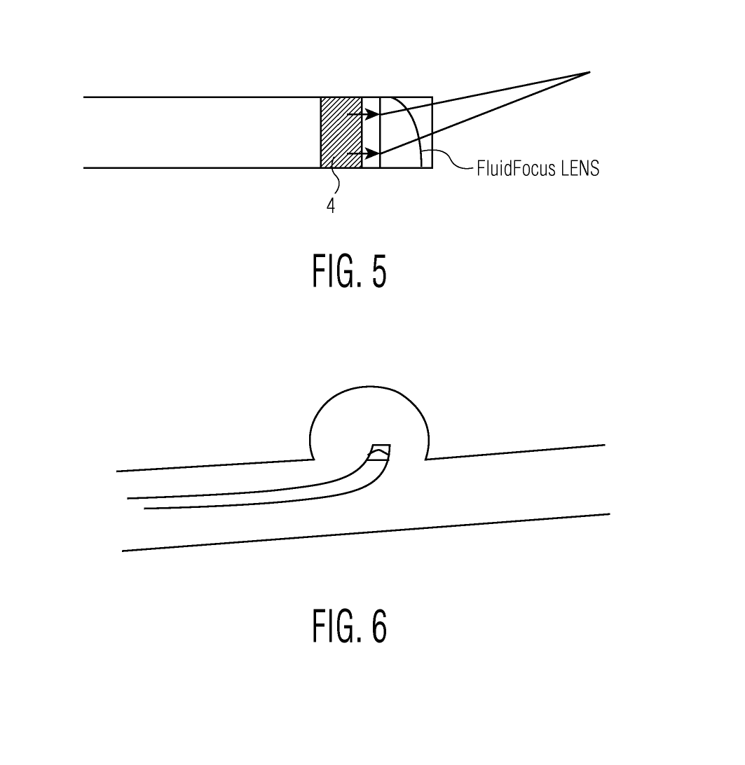

FIG. 6 shows a schematic view of a catheter with ultrasound transducer and variable focus lens guided into the aneurysm.

The invention herein is based on a catheter having ultrasound capability and utilizes a variable focus lens system that allows real-time blood flow read out performed with endovascular ultrasound transducer located proximally to the targeted anatomy or in the anatomy itself In one embodiment of the invention, the endoscope is a single transducer on the tip of a catheter in combination with a variable focus lens system, characterized in that depending on the state of the meniscus (i.e.: the focal length of the lens) the system produces a focal spot along the longitudinal axis of the catheter, or alternatively at a spot substantially perpendicular to the axis of the catheter. By doing Doppler velocity detection at such different positions, this invention allows a 3D velocity map to be constructed for the blood flow, both in the aneurysm pouch or cavity, as well as in the blood vessel or artery, itself.

The new imaging technology solves the following problems associated with prior disclosed devices:

Detection and display of the blood flow speed and blood flow pattern around the probe—both in the parent vessel as well as aneurysms—with high accuracy and high reproducibility

Detection of the blood flow speed and blood flow pattern at different spots inside the aneurysm as well as in the parent vessel proximally and distally of the aneurysm with high accuracy and high reproducibility

Detection of blood flow and blood flow disturbances in the vessel portions with accumulated intraluminal plaque prior to the aneurysm location in order to understand influence of the flow impairments on the aneurysm formation.

Potentially as a predictive method for determination of the stenotic portions and vessel segments where an aneurysm is expected to occur due to the deviate blood flow pattern

FIG. 2 shows an example of how to focus and steer ultrasound using a variable focus lens system. Such lens systems are well known to one skilled in the art, for example, as disclosed in International publication Number WO 2004/051323 A1 published on Jun. 17, 2004.

According to an embodiment of the invention, the catheter has a variable focus lens system placed on its distal tip, on top of an ultrasound transducer. FIG. 3 represents such an embodiment of the invention. The catheter 1 is equipped with an ultrasound transducer 2 placed at the tip 4, from which a non-focused parallel ultrasound beam 2 is emitted into the forward direction. The lens placed on the transducer allows the user to select the focal point for the ultrasound.

When a small radius of curvature is chosen (i.e.: a relatively strong lens with a short focal length), the ultrasound substantially is reflected on the mirror. As a result, in this mode one focuses the ultrasound intensity into a spot perpendicular to the axis of the catheter. This will allow one to focus into an aneurysm pouch and thereby measure Doppler motion signals of blood flowing in the pouch.

When a large radius of curvature is chosen (i.e.: a relatively weak lens with a long focal length), the ultrasound is not significantly reflected on the mirror, because the majority of the intensity will pass above or below the mirror. As a result, in this mode one focuses the ultrasound intensity into a spot along the axis of the catheter. This will allow one to focus into the artery itself and thereby measure Doppler motion signals of blood flowing in the artery. Note that in this case, some of the ultrasound energy will be reflected off the lens in the central part of the beam path, but this needs not be a point of major concern. In fact, by blocking out the central part, the spot size in the focus will be smaller than what it would normally have been when the central part of the beam had also contributed to the image produced. Thus, the endoscope and methodology disclosed herein can be used to dynamically assess the blood flow near an aneurysm during an endovascular procedure.

In another aspect of the invention herein, the catheter utilizes at least one transducer to produce the ultrasound and a variable focus lens system including beam steering, which is capable of focusing ultrasound at various positions both on and off the acoustical axis. With this focused ultrasound we can probe the interior shape of the aneurysm. We can measure the change in blood flow during the procedure by using the Doppler effect. Furthermore, since the shape of the aneurysm can be determined, the position of the catheter tip with respect to the aneurysm and the artery can be determined. According to the systems and methodology disclosed herein the blood flow measurement near and in the aneurysm, 3D imaging of the interior of the aneurysm and precise determination of the position of the catheter tip inside the aneurysm (allowing precise placing of the vaso-embolization material, including vaso-occlusive coils) can be attained, thereby overcoming the problems already discussed regarding prior art devices.

The catheter and methodology according to the invention herein solve the following clinical problems:

Accurate placement of the catheter tip inside the aneurysm pouch in order to allow for safe delivery of the embolization material (either fluid embolization material or coils)

Accurate steering of the catheter in complicated parent vessel and aneurysm anatomy to the aneurysm pouch (this applies to fusiformed multilobulated aneurysms and complicated parent vessels)

Monitoring of the coils delivery during the course of the intervention

Repositioning of the catheter tip in order to release coils in the aneurysm pouch space that is still not filled with the coils

Detection of the misplaced coils that are in danger of being released to the parent vessel blood stream

Accurate positioning of the endovascular material used to protect coils from being dislocated out of the aneurysm (various balloons, bridge devices etc)

Visual assessment of the coils compaction rate during the intervention

Detection and visualization of the thrombus location inside the aneurysm pouch as well as detection of the thrombus dislocation during the course of intervention (caused by the user initiated coil manipulation)

Monitoring of the final coil cast position with respect to the parent vessel once the intervention is completed, in order to allow for undisturbed blood flow in the parent arteries and creation of the membrane formation between the parent vessel and the coiled aneurysm

According to an embodiment of the invention, the catheter has a variable focus liquid lens system placed at the catheter distal end tip, on top of an ultrasound transducer. FIG. 4 shows the layout of the lens capable of steering and focusing ultrasound, which represents the basic embodiment. A schematic drawing is shown of a liquid lens that is capable of deflection or beam steering in (A), and deflection or beam steering in combination with focusing in (B) of a light beam or wave. In a similar way, ultrasound waves can be focused and/or steered. In FIG. 4, (C) and (D) show photographs of a constructed liquid lens corresponding to the beam steering and foucusing shown ,respectively, in (A) and (B). The electrical contacts are on the bottom part of the sidewalls. As is shown in FIG. 5, the catheter is equipped with an ultrasound transducer 4 placed at the tip, from which a non-focused parallel ultrasound beam is emitted into the forward direction of the catheter end. The lens (referred to as FluidFocus lens) placed on the transducer allows the user to select the focal point for the ultrasound in the three-dimensional space. This catheter allows 3D ultrasound imaging of the surrounding by scanning the ultrasound spot. Furthermore, by detecting the Doppler shift it is also possible to detect blood flow at the focal point of the ultrasound beam. By steering the catheter it is possible to enter the aneurysm as is shown in FIG. 6. In this way it is possible to determine the interior shape of the aneurysm and to measure the blood flow. It is also possible to monitor the placing of the coils.

Furthermore, this invention allows the interventional physician to determine the point at which further coil placement is ineffective, e.g. when:

the blood velocity in the aneurysm has decreased to below the thromboses-threshold,

the compaction rate of the coils has ensured a good packing rate, the water hammering effect of the pulsatile blood flow is such as to lower the risk of rebleeding.

With regard to embolization materials that can be used in closing the aneurysm to further blood flow within the aneurysm cavity, various substances, for example:

polymers and other materials, which promote formation of an embolus or occlusion within the aneurysm have been reported and are well known. Vaso-occlusive

coils or packing coils used in treating aneurysms, are generally preferred as an embolization material for delivery and placement within the aneurysm during a surgical intervention. Such coils are well known in the art, for example as disclosed in US Patent Publication 2005/0192618 A1 published on Sep. 1, 2005. The coil wires can be made, for example, of such metals as gold, tungsten and preferably, platinum.

Acoustic variable-focus lenses and means for rapidly adjusting the focal length thereof are disclosed, for example, in PCT publication WO 2005/122139. This publication teaches that preferably, the two fluid media or liquids of the lens have substantially equal densities. Then, the displacement of the part of the boundary is independent of gravitation, and thus independent of the orientation of the lens system. When the two fluid media are not miscible with each another, the boundary is a contact meniscus between the two fluid media. In this case, no wall is placed between both fluid media. Alternatively, the boundary between the different liquids comprises an elastic film. Such film prevents both fluid media from mixing with each another, and it can be stretched by relatively small forces. The lens may also comprise another elastic film, the two elastic films being arranged to hold one of the two fluid media at two respective locations of a path of the acoustic waves. A higher power value of the lens can thus be achieved.

The means for applying the force directly onto at least part of one of the fluid media can be of several types. According to a first type, a first one of the two fluid media comprises a polar and/or electrically conductive liquid substance, and the force applying means comprise an electrode arranged to apply an electric force onto at least part of said first fluid medium. Such means are adapted for electronically controlling the displacement of the boundary. Very rapid variations of the focal length of the acoustic lens can thus be obtained. The electric force is applied advantageously on a part of the first fluid medium which is adjacent the boundary. Then the whole quantity of first fluid medium may be reduced.

According to a second type, the force applying means comprise a movable body contacting said part of the fluid medium. In an optimized embodiment of this type, the movable body may comprise a wall of a vessel containing said part of the fluid medium.

While the present invention has been described with respect to specific embodiments thereof, it will be recognized by those of ordinary skill in the art that many modifications, enhancements, and/or changes can be achieved without departing from the spirit and scope of the invention. Therefore, it is manifestly intended that the invention be limited only by the scope of the claims and equivalents thereof.

1. A catheter comprising:
at least one ultrasound transducer for emitting ultrasound waves;
a variable-focus lens system for variably focusing ultrasound waves emitted by the transducer at various positions;
optionally, a mirror for selectively reflecting the ultrasound waves to a desired position; and
means for translating focused ultrasound waves from the lens system into visual output and communicating the visual output to an operator or device for viewing.

2. The catheter of claim 1 wherein the lens system has at least two liquids with a boundary formed therebetween, and means for applying a force directly onto at least a part of one of the liquids so as to selectively induce a displacement of part of the boundary and thereby vary the focal point of the lens system.

3. The catheter of claim 1 wherein the mirror is positioned in a plane that is at a 45 degree angle relative to a longitudinal axis of the catheter and of a smaller size relative to the size of the lens system.

4. The catheter of claim 1 wherein the tissue of interest is a blood vessel having an aneurysm and the catheter has means for measuring the velocity of blood flow at one or more locations in the vessel and/or the aneurysm and for communicating measured information to an operator or device so as to be viewable.

5. The catheter of claim 4 further comprising means for delivering embolization material into the cavity of the aneurysm, wherein the embolization material is capable of causing an occlusion to form in the aneurysm cavity so as to at least affect, reduce or substantially prevent blood flow into the aneurysm cavity.

6. The catheter of claim 5 wherein the embolization material includes at least one vaso-occlusive wire coil.

7. The catheter of claim 6 wherein the coil is at least substantially made of platinum.

8. A method of viewing a tissue of interest inside of a patient, the method comprising:
placing a catheter having ultrasonic capability so that an end thereof is situated at or near a tissue of interest, the catheter having at least one ultrasound transducer for transmitting ultrasound waves at least substantially along a longitudinal axis in a generally forward direction relative to the end, and for receiving resultant echoes of the ultrasound waves reflected by the tissue of interest, a variable-focus lens system capable of variably focusing the ultrasound waves emitted by the transducer at various positions, optionally, a mirror located so that the emitted ultrasound waves from the lens system are either reflected from the mirror and focused at a position substantially perpendicular to the longitudinal axis; or the emitted ultrasound waves from the lens system substantially bypass the mirror in unreflected form and are focused at a position downstream of the transducer, the lens system and the mirror in the generally forward direction along the longitudinal axis, and imaging means for translating the received echoes of the ultrasound waves reflected by the tissue of interest into images and communicating the images to an operator or device so as to be viewable;
transmitting ultrasound waves from the transducer;
variably focusing the emitted ultrasound waves with the lens system at various positions of the tissue of interest; and
receiving the resultant echoes of the ultrasound waves; and
translating the received echoes of the ultrasound waves into images and communicating the images to an operator or device.

9. The method of claim 8 wherein the lens system has two liquids with a boundary therebetween, and means for applying a force directly onto at least a part of one of the liquids so as to selectively induce a displacement of part of the boundary and thereby vary the focal point of the lens system.

10. The method of claim 8 wherein the mirror is positioned in a plane that is at a 45 degree angle to the longitudinal axis and of a smaller size relative to the size of the lens system.

11. The method of claim 8 further comprising measuring the blood flow velocity and blood flow pattern in a blood vessel and/or an aneurysm associated with the blood vessel, wherein the catheter has means for measuring the velocity of blood flow at one or more locations in the vessel and/or the aneurysm and communicating measured information to an operator or device so as to be viewable.

12. The method of claim 11 further comprising delivering embolization material to the cavity of the aneurysm, wherein the catheter has delivery means for delivering embolization material to the cavity of the aneurysm, the embolization material being capable of causing an occlusion to form in the aneurysm cavity so as to at least affect, reduce or substantially prevent blood flow into the aneurysm cavity.

13. The method of claim 12 wherein the embolization material includes at least one vaso-occlusive wire coil.

14. The method of claim 13 wherein the coil is at least substantially made of platinum.

15. The method of claim 8 wherein the tissue of interest is a blood vessel and the catheter has means for measuring the velocity of blood flow at one or more locations in the vessel and communicating the measured information to an external device or operator.

16. A method of monitoring, during a surgical intervention, the placement of one or more vaso-occlusive wire coils in a blood vessel aneurysm , the method comprising:
placing a catheter having both ultrasound capability and coil delivery capability inside a blood vessel at least near to an aneurysm, the catheter including (i) a variable-focus lens system for variably focusing ultrasound waves at various positions, (ii) optionally, a mirror for selectively reflecting the ultrasound waves to a target position, (iii) means for translating received echoes of the ultrasound waves reflected by the aneurysm and blood vessel into images and communicating such images to a device or operator, (iv) means for measuring blood flow velocity and blood flow pattern in a blood vessel and/or an aneurysm associated with the blood vessel, and(v) means for delivering one or more coils into the cavity of the aneurysm;
providing ultrasound waves via at least one transducer;
variably focusing ultrasound waves via the lens system at various positions of the blood vessel and aneurysm cavity; and
receiving, via the at least one transducer, information relative to resultant echoes of the ultrasound waves and blood velocity measurements; and
translating received information into output and communicating the output to a device or operator so to enable the monitoring of a coil placement.
